(19) United States
(12) Patent Application Publication
(10) Pub. No.: US 2026/0137636 A1
(43) Pub. Date: May 21, 2026

(54) TUMOR-TARGETING THIOCOLCHICINE NANOEMULSION

(71) Applicant: Henry Smith, Temecula, CA (US)

(72) Inventor: Henry Smith, Temecula, CA (US)

(21) Appl. No.: 18/950,839

(22) Filed: Nov. 18, 2024

(51) Int. Cl. A61K 31/167; A61K 31/337; A61K 31/4745; A61K 31/513; A61K 31/704; A61K 38/12; A61K 47/68

(52) U.S. Cl. CPC A61K 31/167; A61K 31/337; A61K 31/4745; A61K 31/513; A61K 31/704; A61K 38/12; A61K 47/6851

(57) ABSTRACT

This invention teaches a tumor-targeting thiocolchicine nanoemulsion wherein the nanodroplets comprising the nanoemulsion are coated with an antinuclear antibody (ANA-nanoemulsion). The nanodroplets are less than 200 nm and will enter the tumor via the Enhanced Permeation and Retention Effect (EPR). Once inside the tumor the antinuclear antibody coating the nanodroplets will bind to extracellular nuclear material released from dead cells and anchor the nanodroplets within the tumor where the drug is released for maximum effect. As all solid tumors have areas of necrosis this means that the ANA-nanoemulsion can target and treat all solid tumors regardless of the tumor type. The ANA-nanoemulsion is formulated to have a phase transition temperature above 8 C. When cooled to below 8 C the ANA-nanoemulsion converts into a suspension of solid lipid nanospheres (ANA-nanospheres) suitable for storage at 4 C or below.

### CROSS-REFERENCE TO RELATED APPLICATIONS

None

### STATEMENT REGARDING FEDERALLY SPONSORED RESEARCH OR DEVELOPMENT

None

### BACKGROUND OF THE INVENTION

As early as 1500 BC a plant extract made for the autumn crocus (Colchicum autumnale) was used to treat joint swelling. Since that time, it has been used to treat various conditions, and even today the active component—colchicine is used as a treatment for gout and familial Mediterranean fever.

Researchers observed that colchicine inhibits cell division by disrupting the microtubules responsible for the correct alignment of the chromosomes on the mitotic spindle and this disorganization of the chromosomes results in the death of the cell. The observation that colchicine was a mitotic poison suggested that it could be used as a cancer drug. However, the level of colchicine required to treat cancer was also unacceptably toxic to healthy cells and further efforts to use colchicine as a cancer drug were largely discontinued. Research efforts continued to develop analogs of colchicine that investigators hoped would retain cytotoxicity to cancer while causing less harmful effects to normal tissues. One of the colchicine analogs developed is thiocolchicine.

Thiocolchicine (N-[(7S)-1,2,3-trimethoxy-10-methylsulfanyl-9-oxo-6,7-dihydro-5H-benzo[a]heptalen-7-yl] acetamide) is an antimitotic alkaloid that binds to microtubules and inhibits tubulin polymerization and induces apoptosis. Thiocolchicine MW 415.5 logP 2.7 is a lipid soluble drug that can be incorporated into an oil-in-water nanoemulsion.

This invention teaches a formulation of thiocolchicine incorporated in an oil-in-water nanoemulsion wherein the nanodroplets comprising the nanoemulsion are coated with a unique antinuclear antibody that can target all solid tumors. There is no teaching in the prior art of a tumor-targeting biopharmaceutical that has the same or similar form and function to the tumor-targeting thiocolchicine nanoemulsion taught in the instant invention.

### SUMMARY OF THE INVENTION

This invention teaches that all solid tumors have areas of necrosis composed of dead cells that have released their nuclear material into the extracellular medium; and that this feature can be exploited to develop a tumor-targeting drug delivery system that can target all solid tumors. This invention teaches a tumor-targeting antinuclear antibody-linked thiocolchicine nanoemulsion (ANA-nanoemulsion) wherein thiocolchicine is incorporated into the nanodroplets comprising the nanoemulsion. The nanodroplets are then coated with an antinuclear antibody. The nanodroplets are made to be of a uniform size that is under 200 nm in diameter. This will allow the nanodroplets to extravasate through the enlarged endothelial pores of the leaky blood vessels supplying the tumor and enter the tumor by a process known as the “Enhanced Permeability and Retention” effect (EPR). Once inside the tumor the antinuclear antibody attached to the nanodroplets will bind to the extracellular nuclear material released from dead cells, and thus anchor the nanodroplets within the tumor where the drug is released to kill surrounding tumor cells. As all solid tumors, irrespective of their cancer cell type, have areas of necrosis filled with dead cells that have released their nuclear material into the extracellular medium, this means that the ANA-nanoemulsion disclosed in this invention can target and treat all types of solid tumors.

Further, the ANA-nanoemulsion is made to have a phase transition temperature that is above 8 C such that upon cooling to below its phase transition temperature the ANA-nanoemulsion will convert into a suspension of solid lipid nanospheres trapping the thiocolchicine within the solid lipid matrix of the nanosphere. The thiocolchicine incorporated solid lipid nanospheres (ANA-nanospheres) can now be stored at 4 C or below for an extended period.

### DRSCRIPTION OF THE INVENTION

This invention teaches the form and function of a tumor-targeting nanoemulsion wherein the cancer drug thiocolchicine is incorporated in an oil-in-water nanoemulsion, and wherein the nanodroplets comprising said nanoemulsion are coated with an antinuclear antibody. In this invention the term “drug” refers to the cancer drug-thiocolchicine, when it is used alone or when it is incorporated into an ANA-nanoemulsion as disclosed in this invention. The term “incorporated into a nanoemulsion” includes the drug being present in the oil component, and/or in the phospholipid component, and/or associated with the surfactant and/or cosurfactant layer of the nanodroplets comprising the nanoemulsion. The term “tumor-targeting antibody” is used to describe the antinuclear antibody (ANA) and/or the Fab binding fragment of said antibody that can bind to the extracellular nuclear antigens present in the necrotic regions of tumors.

To prepare the ANA-nanoemulsion the drug is co-dissolved in an organic solvent such as chloroform/methanol with a mixture of oils, phospholipids, surfactant and other components, and heated to above the phase transition temperature of any of its constituents. The dissolved mixture of constituents is then dried under vacuum in a rotary evaporator (Hei-VAP Heidolph USA) to remove the solvent, and the oily residue is then hydrated with heated 0.1 percent Tween 80 in distilled water to form an emulsion. The emulsion is homogenized, sonicated and extruded thru a series of membranes of decreasing pore sizes using a commercial extruder (EmulsiFlex-05, Avestin, Canada) to form a nanoemulsion. The nanodroplets comprising the nanoemulsion are made to be of uniform size below 200 nm in diameter, and preferably to be about 150 nm in diameter. Throughout the process the temperature is maintained above the phase transition temperature of the lipids used to prepare the nanoemulsion.

The nanoemulsion described above is then converted into a tumor-targeting nanoemulsion by coating the nanodroplets with the Fab fragment of an antinuclear antibody (ANA-nanoemulsion). The ANA-nanoemulsion is then cooled to below its phase transition temperature whereupon it converts into a suspension of solid lipid nanospheres (ANA-nanospheres) trapping the thiocolchicine within the solid lipid matrix of the nanosphere. The ANA-nanospheres can be stored at 4 C or below for an extended period without the nanospheres coalescing or the drug leaking out of the nanospheres.

### Example 1. Preparation of Antinuclear Antibody-Linked Thiocolchicine Nanoemulsion (ANA-Nanoemulsion)

The following example is provided to illustrate a typical method of preparing the ANA-nanoemulsion taught in this invention. Those of skill in the art will be cognizant that there are a variety of other methods of preparing a nanoemulsion that can be used in lieu of the method described herein. Therefore, the precise method described in this example is not to be construed as any limitation on the method of preparing said nanoemulsion. Instead, all methods of preparing a nanoemulsion to arrive at the same result disclosed in this invention are considered to lie within the spirit and scope of this invention.

This invention teaches the preparation of the tumor-targeting ANA-nanoemulsion is performed in two stages. The first stage is the process of incorporating thiocolchicine into the nanodroplets comprising the nanoemulsion. The second stage is the process of coating the thiocolchicine incorporated nanodroplets with the tumor-targeting antinuclear antibody.

### First Stage. Preparation of a Thiocolchicine Incorporated Nanoemulsion

To prepare the nanoemulsion the ingredients used are: 1,000 mg coconut oil; 20 mg alpha-tocopherol (Vitamin E oil); 400 mg hydrogenated soy phosphatidylcholine (HSPC): 100 mg polyethylene glycol-derivatized distearoyl-phosphatidylethanolamine i.e. DSPE-PEGn where n is a molecular weight of 2,000 daltons (DSPE-PEG 2000); 10 mg DSPE-PEG 2,000-Maleimide (DSPE-PEG 2,000-MAL); and 20 mg thiocolchicine. The ingredients are mixed and heated to 70 C and an organic solvent composed of chloroform/methanol is added to ensure that all the constituents are dissolved. The solvent is then removed under vacuum and heating at 70 C for several hours using a rotary evaporator (Hei-VAP Heidolph USA) leaving an oily residue. The oily residue is hydrated with 100 ml of 0.1 percent Tween 80 in distilled water heated to 70 C and shaken and homogenized for 30 seconds using a handheld homogenizer to produce an emulsion. The emulsion is then sonicated for 15 minutes in a heated waterbath-sonicator (VWR) and then repeatably extruded using a commercial extruder (EmulsiFlex-05, Avestin, Canada) through stacked membranes of decreasing pore sizes i.e. 400 nm, 200 nm, and 100 nm until a uniform sized nanoemulsion is formed wherein the nanodroplets are below 200 nm in diameter, and preferably about 150 nm in diameter. A temperature of 70 C is maintained throughout the whole process.

### Second Stage. Coating the Thiocolchicine Incorporated Nanodroplets with the Antinuclear Antibody

The thiocolchicine nanoemulsion is converted into a tumor-targeting nanoemulsion by coating the nanodroplets comprising the nanoemulsion with the Fab fragment of the antinuclear antibody. The antinuclear antibody disclosed in this invention is obtained from the pooled sera of patients with Systemic Lupus Erythematosus (SLE).

In this invention the antinuclear antibody obtained from patients with SLE is preferred because it comprises a spectrum of polyclonal antibodies that can bind to a variety of different nuclear antigens. Serum from these patients is collected and pooled. A purified antinuclear antibody fraction is obtained using established laboratory methods such as ammonium sulphate precipitation and affinity chromatography. The Immunoglobulin fraction of the pooled sera is precipitated out using 33 percent cold saturated ammonium sulphate and the precipitate dissolved in phosphate buffered saline (PBS). A nuclear extract is prepared from mouse liver cells, and the nuclear extract is covalently linked to cyanogen bromide activated cross-linked Sepharose beads in an affinity column. The immunoglobulin fraction is flowed through the column and the bound antinuclear antibodies are eluted off the column using a glycine-HCl buffer pH 2.5. The pH of the eluate is adjusted to pH 7.0 using NaOH. The purified antinuclear antibody solution was tested for binding activity using an Enzyme Linked Immunosorbent Assay (ELISA) for antinuclear antibody (AccuDiag ELISA, Cortez Diagnostics). The result demonstrated that the purified antinuclear antibody solution showed strong binding activity to the nuclear antigens that was much higher than the positive control.

To covalently link the Fab fragment of the antinuclear antibody to the maleimide site the antinuclear antibody molecule is cleaved into the Fab and Fc fragments using immobilized papain, and the Fc fragment is removed using immobilized Protein A. The Fab fragment is then attached to the exterior of the drug incorporated lipid nanodroplets by incubating the Fab with the nanoemulsion overnight at a temperature that is above the phase transition temperature of the nanoemulsion whereupon the Fab is covalently linked to the maleimide site on the DSPE-PEG2,000-MAL molecule. Maleimide sites that are unconjugated are capped using cysteine. The Fab is thus attached to the exterior of the nanodroplets through the linking DSPE-PEG-maleimide molecule. Any unreacted material is removed using gel-filtration or rapid dialysis against distilled water. The ANA-nanoemulsion is filtered through a 0.22-micron filter (Millipore) and placed in a sterile vial.

The ANA-nanoemulsion is cooled to below its phase transition temperature whereupon it transforms into a suspension of solid lipid nanospheres (ANA-nanospheres) suitable for storage at 4 C or below. The ANA-nanospheres are stored at 4 C in a sealed vial under an inert gas such as nitrogen gas and placed in a light proof container. Upon rewarming to above its phase transition temperature the ANA-nanospheres will convert back into an oil-in-water ANA-nanoemulsion suitable for administration to the patient in need. For prolonged storage a cryoprotectant such as sucrose or trehalose can be added to the suspension of ANA-nanospheres and then lyophilized and stored at −20 C. Upon addition of the correct amount of distilled water and rewarming the lyophilized preparation to above its phase transition temperature it will convert back into an oil-in-water ANA-nanoemulsion suitable for administration to the patient in need.

In one embodiment of this invention the antinuclear antibody is attached to the solid lipid nanospheres. In this method the thiocolchicine incorporated nanoemulsion is prepared as described earlier and then cooled to below its phase transition (e.g. 4 C) whereupon it converts into a suspension of solid lipid nanospheres. The lipid nanospheres are now mixed with the Fab fragment of the antinuclear antibody and incubated overnight at 4 C whereupon the Fab fragment is covalently linked to the maleimide site on the DSPE-PEG2,000-MAL molecule. Maleimide sites that are unconjugated are capped using cysteine. The Fab is thus attached to the exterior of the solid lipid nanospheres through the linking DSPE-PEG-maleimide molecule. Unreacted material is removed using gel-filtration or rapid dialysis against distilled water. The ANA-nanospheres are stored at 4 C or lyophilized and stored at −20 C as described earlier. Upon rewarming to above its phase transition temperature the ANA-nanospheres will convert back into an oil-in-water ANA-nanoemulsion suitable for administration to the patient in need.

The ANA-nanoemulsion disclosed in this invention has the following advantages. When administered to the patient the drug being incorporated in the nanoemulsion is protected from being detoxified by the liver or filtered out by the kidneys. The presence of the long chain PEG polymers coating the nanodroplets will also provide steric hindrance to reduce their uptake by the reticuloendothelial immune system in the liver and spleen. There is thus more drug bioavailable for a longer period of time compared to the predicate free drug. Further, the nanodroplets are too large to pass through the endothelial pores of normal blood vessels into healthy tissues to cause harm. Tumors however are supplied by blood vessels that have abnormally enlarged endothelial pores that can reach 400 nm in diameter. The nanodroplets comprising the ANA-nanoemulsion being smaller than 200 nm can extravasate through these enlarged pores and enter the tumor. This mechanism of passive accumulation of nanoparticles within the tumor is known as the “Enhanced Permeability and Retention effect” (EPR).

This invention teaches that the retention of the nanodroplets within the tumor is further enhanced because the antinuclear antibody coating the nanodroplets will bind to the extracellular nuclear material present in the necrotic regions of the tumor and thus anchor the nanodroplets in those regions of the tumor. There is therefore an accumulation of the ANA-nanoemulsion within the tumor. The thiocolchicine incorporated in the nanoemulsion is now released from the nanoemulsion and will diffuse out and kill the surrounding tumor cells. The end-result of this invention is that more drug reaches the tumor to inhibit tumor growth, and less drug reaches normal tissues to cause harm.

There are many different oils, phospholipids and surfactants that can be used to make the ANA-nanoemulsion. For example, in one embodiment of this invention the oil component of the nanoemulsion is a mixture of coconut oil and Vitamin E oil; the phospholipids are hydrogenated soy phosphatidylcholine (HSPC); polyethylene glycol-derivatized distearoyl-phosphatidylethanolamine (DSPE-PEGn where n is a molecular weight of 2,000 daltons or above; e.g. (DSPE-PEG 2,000); and DSPE-PEGn with a maleimide group attached to the distal end of the polymer chain e.g. DSPE-PEGn-Maleimide (DSPE-PEG2,000-MAL).

The nanodroplets comprising the nanoemulsion prepared in this way will have the following structure. There is a spherical oil core surrounded by a layer of phosphatidylcholine with the tails of the phosphatidylcholine molecule embedded in the oil and the polar heads of the molecule oriented to the exterior. Also making up part of the lipid layer is the DSPE-PEG2,000 where the lipid component of the molecule is embedded in the surface lipid layer with the PEG2,000 polymer chains extending out into the surrounding aqueous medium. Also making up part of the lipid layer is the DSPE-PEG2,000-MAL where the lipid component of the molecule is embedded in the surface lipid layer with the PEG2,000-MAL polymer chains extending out into the surrounding aqueous medium. The PEG chains provide steric hinderance between the nanodroplets and help prevent them from aggregating during storage. When the ANA-nanoemulsion is injected into the patient the PEG polymer chains also provide steric hinderance to prevent the nanoemulsion from uptake by the reticuloendothelial system (RES) of the patient.

The DSPE-PEG2,000-MAL function is to link the Fab fragment of the antinuclear antibody to the exterior surface of the nanodroplet. It is incorporated into the nanodroplet with the lipid component of the molecule embedded in the surface lipid layer with the PEG2,000-MAL polymer chains extending out into the medium and bearing the maleimide site at the distal end of the polymer chain. The Fab fragment of the antinuclear antibody is then covalently attached to the maleimide site leaving the binding site of the Fab free to bind to the extracellular nuclear antigens present in the necrotic areas of tumors.

Those of skill in the art will know that other oils can be substituted for the coconut oil and/or the Vitamin E oil without departing from the spirit and scope of this invention. The group of oils that can be used includes coconut oil, castor oil, corn oil, canola oil, soybean oil, peanut oil, olive oil, sunflower oil, palm oil, nutmeg oil, primrose oil, fish oil, Vitamin E oil, mineral oil and triglycerides. The natural oils can be hydrogenated which results in them having a higher phase transition temperature than the natural untreated oil. For example, natural coconut oil has a phase transition temperature of 26 C whereas hydrogenated coconut oil has a phase transition temperature of 36 C.

In one embodiment of this invention a single oil such as natural coconut oil which has a phase transition temperature of 26 C, or hydrogenated coconut oil which has a phase transition temperature of 36 C, is used in the nanoemulsion. However, to obtain a particular desired phase transition temperature for the nanoemulsion a mixture of two or more oils with differing phase transition temperatures is used. Combining different ratios of a high phase transition temperature oil with a low phase transition temperature oil will yield a spectrum of nanoemulsions with different phase transition temperatures from which the nanoemulsion with the desired [phase transition temperature can be selected.

In one embodiment of this invention the ANA-nanoemulsion has a phase transition temperature that is a value that falls within the range of 9 C to 36 C such that below its phase transition temperature it transforms into a suspension of solid lipid ANA-nanospheres suitable for storing at 4 C or below. When required the ANA-nanospheres can be warmed to above their phase transition temperature whereupon the ANA-nanospheres will transform into an oil-in-water ANA-nanoemulsion before administration into the cancer patient. The advantage of storing solid lipid nanospheres rather than storing an oil-in-water nanoemulsion is that there is no leakage of the drug out of the solid lipid nanospheres during storage; and the solid lipid nanospheres do not coalesce or aggregate during storage.

In one embodiment of this invention the ANA-nanoemulsion has a phase transition temperature that is a value that falls within the range of 38 C to 45 C such that below its phase transition temperature it transforms into a suspension of solid lipid nanospheres suitable for storing at 4 C or below. When required, the ANA-nanospheres can be warmed to room temperature or to a body temperature of 37 C before administration into the cancer patient. However, note that as the ANA-nanospheres have a phase transition temperature that is above a body temperature of 37 C the ANA-nanospheres remain as a suspension of solid lipid nanospheres when it is administered to the patient. Administering the drug incorporated nanocarrier in the form of solid lipid nanospheres may prolong drug release in vivo compared to drug release from an oil-in-water nanoemulsion. This may result in a better safety and efficacy profile compared to using a nanoemulsion.

There are a large variety of methods described for preparing nanoemulsions and the means whereby lipid soluble cancer drugs can be incorporated into said nanoemulsions. For example, drug loaded nanoemulsions can be prepared using homogenization, or sonication, or shearing, or self-emulsifying, or microfluidizing methods. They can also be prepared using a combination of methods such as homogenization and sonication to prepare a coarse emulsion followed by extruding the coarse emulsion through nanosized pores to produce a nanoemulsion. These and other methods of preparing a nanoemulsion are known to those of skill in the art and are therefore considered to be within the scope of this invention.

In order to obtain a stable nanoemulsion one or more emulsifying agents and surfactants are included in the formulation. The emulsifying agent is selected from a list that includes: egg phosphatidylcholine (EPC), soy phosphatidylcholine (SPC), hydrogenated soy phosphatidylcholine (HSPC), phosphatidylethanolamine (PE), phosphatidylglycerol (PG), phosphatidylinsitol (PI), monosialoganglioside and sphingomyelin (SPM); the derivatized vesicle forming lipids such as poly(ethylene glycol)-distearoylphosphatidylethanolamine (DSPE-PEG2000 and DSPE-PEG5000), polyethyleneglycol-ceramides (CER-PEG), distearoylphosphatidylcholine (DSPC), and dimyristoylphosphatidylcholine (DMPC).

In this invention the non-ionic surfactants are preferred and include: polysorbate 80 (Tween 80), polysorbate 20 (Tween 20), Poloxamer 188, Brij 35 and the like. The surfactants may be used singly or in combination. Optionally other co-surfactants such as short-chain alcohols e.g. ethanol may be added to the formulation to reduce the size of the nanodroplets in the nanoemulsion.

The antinuclear antibodies disclosed in this invention were obtained from patients with SLE. There are several advantages to using said antibodies. They are present in high titers in SLE patients and can be purified using conventional methods for purification such as ammonium sulphate precipitation and affinity chromatography. Also being human derived they will not elicit an immune response when administered to a patient. These are polyclonal antibodies and will bind to a variety of nuclear antigens such as double-stranded DNA (dsDNA), single-stranded DNA (ssDNA), deoxynucleoprotein (DNP), soluble nuclear proteins (sNP), and extractable nuclear antigen (ENA) including Smith (Sm) antigen and ribonucleoprotein (RNP). This will provide more opportunities for the polyclonal antinuclear antibodies to bind to a variety of nuclear antigens present in the extracellular nuclear material found in necrotic areas of tumors.

There are methods of developing antinuclear antibodies using monoclonal and genetic engineering techniques that are known to those of skill in the art. Said monoclonal or recombinant antibodies can be human derived or humanized using procedures known to those of skill in the art. Different monoclonal antibodies can preferably be combined to achieve a broad binding capacity to the different nuclear antigens that are present in the necrotic areas of the tumor. Similarly different recombinant antibodies can preferably be combined to achieve a broad binding capacity to the different nuclear antigens that are present in the necrotic areas of the tumor. Because the combined monoclonal antinuclear antibodies and the combined recombinant antinuclear antibodies are designed to mimic the SLE patient derived antinuclear antibodies disclosed in this invention they are therefore considered to lie within the spirit and scope of this invention.

Those of skill in the art will know that there are also binding agents such as aptamers and binding peptides that are functionally equivalent to antibodies and can be used in lieu of using antibodies. Aptamers are small (i.e. 40-100 bases), synthetic single-stranded oligonucleotides (ssDNA or ssRNA) that can specifically recognize and bind to virtually any kind of target, including ions, whole cells, drugs, toxins, low-molecular-weight ligands, peptides, and proteins. Each aptamer has a unique configuration because of the composition of the nucleotide bases in the chain causing the molecule to fold in a particular manner. Because of their folded structure each aptamer will bind selectively to a particular ligand in a manner analogous to an antibody binding to its antigen. Aptamers are usually synthesized from combinatorial oligonucleotide libraries using in vitro selection methods such as the Systematic Evolution of Ligands by Exponential Enrichment (SELEX). This is a technique used for isolating functional synthetic nucleic acids by the in vitro screening of large, random libraries of oligonucleotides using an iterative process of adsorption, recovery, and amplification of the oligonucleotide sequences. The iterative process is carried out under increasingly stringent conditions to achieve an aptamer of high affinity for a particular target ligand. The use of aptamers as functional binding agents equivalent to the antinuclear antibody disclosed in this invention is therefore considered to lie within the spirit and scope of this invention.

Binding peptides that fold in such a manner that their configuration makes them capable of binding to antigens in a manner that mimics the binding of an antibody to its antigen. There are various well-known methods for preparing synthetic or biological peptide libraries composed of up to a billion different sequences, and for identifying a particular peptide sequence that will target a particular antigen. The use of binding peptides as functional binding agents that are functionally equivalent to the antinuclear antibody disclosed in this invention is therefore considered to lie within the spirit and scope of this invention.

In one embodiment of this invention a second lipid soluble cancer drug is combined with the thiocolchicine and incorporated in the tumor-targeting nanoemulsion. Those of skill in the art of cancer chemotherapy typically administer multiple different cancer drugs in treating the cancer patient. The cancer drugs selected will depend on the type and stage of the tumor, and the age, sex, and clinical status of the patient. Each cancer drug is also typically administered separately on a different day according to a defined schedule to avoid the unacceptable toxicity to the patient if they are administered together. However, because of the improved safety that results when a cancer drug is incorporated in a nanocarrier such as a nanoemulsion it is possible to combine several cancer drugs within a nanoemulsion without compromising safety to the patient. This invention teaches the following lipid soluble drugs that can be combined with thiocolchicine in the ANA-nanoemulsion. The lipid soluble drug is selected from the group that includes: dactinomycin MW 1,255, logP 3.2; docetaxel MW 808, logP 2.4; irinotecan MW 677, logP 4.4; daunorubicin MW 528, logP 1.8; and carmofur MW 257, logP 2.6.

The method of combining two lipid soluble drugs i.e. thiocolchicine and a second lipid soluble drug in the ANA-nanoemulsion is the same as that described in Example 1; except that in addition to incorporating thiocolchicine alone in the nanoemulsion, a second lipid soluble cancer drug is combined with the thiocolchicine and both drugs are incorporated together in the nanoemulsion. The relative amounts of thiocolchicine and the second cancer drug used in the ANA-emulsion will depend on the identity of the second cancer drug selected, and its known safety and efficacy profile. However, because the bioavailability and biodistribution of a drug is significantly different when it is incorporated in a nanocarrier as compared to when it is given as a free drug, each drug combination in the ANA-nanoemulsion will require further testing in vivo before a final formulation can be determined.

This invention teaches incorporating thiocolchicine in a tumor-targeting nanoemulsion. Those of skill in the art would be cognizant that there is ongoing research into developing different analogs of thiocolchicine in the hope that they will be safer and/or more effective in inhibiting tumor growth. Based on the disclosures in this invention, any lipid soluble analog of thiocolchicine that has a similar safety and efficacy profile to that of thiocolchicine and which is incorporated into a tumor-targeting nanoemulsion similar to that disclosed in this invention is considered to lie within the spirit and scope of this invention.

### DISCUSSION

The ANA-nanoemulsion disclosed in this invention is typically administered by intravenous injection or infusion into the cancer patient in need. The size of the nanodroplets is critical when developing targeting biopharmaceuticals that will be administered parenterally. If the nanodroplets are too large i.e. above 200 nm they are rapidly taken up by the reticuloendothelial system (RES) in the liver and spleen and removed from the blood. Smaller nanodroplets below 200nm and preferably about 150 nm or smaller will remain in circulation for a longer period. This increases the time that the drug is bioavailable to enter the tumor and act upon the tumor cells. Also, in this invention the surface of the nanodroplets is coated with extended hydrophilic PEG chains that provide steric hindrance to recognition by the RES and thus allow more of the drug to be bioavailable for a longer period of time.

The nanodroplets will circulate in the blood stream because they are too large to extravasate through the endothelial pores of normal blood capillaries supplying normal healthy tissues. However, tumors are served by blood vessels that have very enlarged endothelial pores that can reach 400 nm or more in diameter. When the nanodroplets reach these “leaky” blood capillaries they can extravasate through the enlarged endothelial pores and into the tumor. Once inside the tumor the Fab will bind to the extracellular nuclear antigens present in necrotic regions of the tumor and thus anchor the nanoemulsion within the tumor where the drug is released to kill the surrounding tumor cells. The end-result is that compared to the predicate free drug the ANA-nanoemulsion of this invention will be more effective against the tumor and with less harm to normal tissues.

The antinuclear antibody used in this invention is unique when compared to all the tumor-targeting antibodies described in the prior art. This is because the antinuclear antibody does not target any cellular receptor or cell-surface marker on the tumor cell. Instead, the antinuclear antibody in this invention targets extracellular nuclear material released from dead cells irrespective of whether the nuclear material came from dead tumor cells or dead normal cells. The sole function of the antinuclear antibody is to anchor the ANA-nanoemulsion within the necrotic regions of the tumor. This results in the accumulation of the drug incorporated ANA-nanoemulsion within the tumor where the drug is released to kill the surrounding tumor cells.

The antinuclear antibody of this invention can target all types of solid tumors regardless of the cell-type from which they originated. This is because all solid tumors have areas of necrosis filled with dead cells and extracellular nuclear material released from dead cells. This extracellular nuclear material is targeted by the ANA-nanoemulsion taught in this invention and will result in the ANA-nanoemulsion accumulating within the tumor where the incorporated cancer drug thiocolchicine is released for maximum effect. Therefore, the ANA-nanoemulsion can target and treat all solid tumors including lung cancer, breast cancer, ovarian cancer, colon cancer, pancreatic cancer, prostate cancer, liver cancer, melanoma, and various sarcomas and carcinomas. This differentiates it from all other tumor-targeting agents that typically target a cell-surface receptor or cell-surface antigen on the tumor cell.

The advantage of having a tumor-targeting drug delivery system that can target all solid tumors regardless of whether the tumor has tumor associated markers or not is obvious. For example, consider the case of “triple-negative breast cancer” where the breast cancer cell lacks the estrogen receptor, the progesterone receptor, and where the epidermal growth factor receptor 2(HER2 ) is not over-expressed. The prognosis for patients with triple-negative breast tumor is very serious because none of the current tumor-targeting drugs are effective against this tumor. However, because triple-negative breast tumors have pockets of dead cells the ANA-nanoemulsion disclosed in this invention can target and treat triple-negative breast cancers. In this context it's important to note that the same ANA-nanoemulsion that can treat triple-negative breast cancer can also target and treat all solid tumors regardless of the tumor type and even if the tumor cell lacks all known tumor-associated markers.

Another important feature in this invention is its teaching of preparing the ANA-nanoemulsion to have a phase transition temperature above 8 C so that it is in the form of solid lipid nanospheres (ANA-nanospheres) when it is stored at 4 C in a refrigerator, or lyophilized and stored frozen at −20 C. The ANA-nanospheres are inherently more stable during storage than an ANA-nanoemulsion. The solid lipid ANA-nanospheres do not coalesce or aggregate during storage. There is also no leakage of the thiocolchicine out of the solid lipid ANA-nanospheres during storage.

It will be obvious to one of skill in the art from the teaching provided in the instant invention that there are various modifications and changes that can be made to the thiocolchicine incorporated ANA-nanoemulsion disclosed in this invention without departing from the spirit and scope of this invention. Said changes are therefore within the scope of this invention.

What is claimed is:

1. A tumor-targeting biopharmaceutical that is a nanoemulsion composed of a) thiocolchicine, b) one or more oils, c) one or more phospholipids, d) DSPE-PEGn where n is a MW above 2,000, e) DSPE-PEGn-Maleimide where n is a MW above 2,000, f) one or more surfactants; and wherein the nanodroplets comprising the nanoemulsion are g) sized to be under 200 nm in diameter, and h) have a phase transition temperature above 8 C, and i) are coated with the Fab fragment of an antinuclear antibody obtained from patients with Systemic Lupus Erythematosus (SLE).

2. A tumor targeting biopharmaceutical according to claim 1 wherein the one or more oils are selected from the group of coconut oil, castor oil, corn oil, canola oil, soybean oil, peanut oil, olive oil, sunflower oil, palm oil, nutmeg oil, primrose oil, fish oil, Vitamin E oil, mineral oil and triglycerides.

3. A tumor-targeting biopharmaceutical according to claim 1 wherein the one or more phospholipids are selected from the group of egg phosphatidylcholine (EPC), soy phosphatidylcholine (SPC), hydrogenated soy phosphatidylcholine (HSPC), phosphatidylethanolamine (PE), phosphatidylglycerol (PG), phosphatidylinositol (PI), monosialoganglioside and sphingomyelin (SPM);
distearoylphosphatidylcholine (DSPC), and dimyristoylphosphatidylcholine (DMPC).

4. A tumor-targeting biopharmaceutical according to claim 1 wherein the surfactant is selected from the group of polysorbate 80 (Tween 80), polysorbate 20 (Tween 20), Poloxamer 188, and Brij 35.

5. A tumor-targeting biopharmaceutical according to claim 1 wherein the phase transition temperature of the nanoemulsion is set to be within a temperature range of 9 C to 36 C.

6. A tumor-targeting biopharmaceutical according to claim 1 wherein the phase transition temperature of the nanoemulsion is set to be within a temperature range of 38 C to 45 C.

7. A tumor-targeting biopharmaceutical according to claim 1 wherein a therapeutic dosage of said nanoemulsion is administered to a cancer patient in need by intravenous injection or infusion.

8. A tumor-targeting biopharmaceutical according to claim 1 wherein in addition to the components listed in claim 1 a second lipid soluble cancer drug is incorporated in said nanoemulsion, and wherein the second cancer drug is selected from the group of dactinomycin, docetaxel, irinotecan, daunorubicin, and carmofur.

9. A tumor-targeting biopharmaceutical according to claim 8 wherein a therapeutic dosage of said nanoemulsion is administered to a cancer patient in need by intravenous injection or infusion.
